(19) United States
(12) Patent Application Publication
(10) Pub. No.: US 2020/0397934 A1
(43) Pub. Date: Dec. 24, 2020

(54) FORMULATION FOR FORMING A DISINFECTING FOAM

(71) Applicant: saniXTREME GmbH & Co. KG, Frankfurt am Main (DE)

(72) Inventor: Marco Hess, Neu-lsenburg (DE)

(21) Appl. No.: 16/767,217

(22) PCT Filed: Nov. 21, 2018
(86) PCT No.: PCT/EP2018/000520

§ 371 Date: May 27, 2020

(30) Foreign Application Priority Data
Nov. 27, 2017 (DE) 10 2017 010 923.0

(51) Int. Cl. A61L 2/235; C25B 1/26; A61K 8/04; A61K 8/20; A61K 8/31; A61K 8/33

(52) U.S. Cl. CPC A61L 2/235; C25B 1/26; A61K 8/046; A61L 2101/08; A61K 8/315; A61K 8/33; A61K 8/20

(57) ABSTRACT

A formulation for providing a disinfecting foam on the basis of an electrochemically activated aqueous saline solution enabling the dispensation as a foam while the microbicidal effectivity the electrochemically activated saline solution is maintained is to be provided. To this end, the formulation comprises at least one surfactant foam carrier component and at least one antimicrobially effective component on the basis of a electrochemically activated aqueous saline solution, the formulation having a content of at least 10 ppm of a free chlorine species selected from the group comprising hydrochlorous acid, hypochlorite ions, sodium hypochlorite or dissolved chlorine gas, and exhibiting a vibration band associated with a C—F vibration in the infrared spectrum.

The invention relates to a formulation for providing a disinfecting foam. It further relates to a disinfecting foam obtainable by such formulation.

Electrochemically activated aqueous saline solutions produced according to a so-called “ECA” (“electrochemical activation”) method, also referred to as anolytes, are highly effective disinfectants having exceptional microbicidal properties. Such a solution can, in particular, be obtained by means of the electrolysis of sodium chloride solutions. Depending on the selected dilution, such an anolyte can be used, for example, for applications for surface disinfection, e.g. of worktops, tables, floors, but also for cold disinfection actions, for the removal of von microorganisms in agriculture, for washing laundry, in applications in swimming baths, or even as a prophylaxis against foot fungus. When required, and in an applicable formulation, however, such an anolyte can also be used as an active agent for combating microbes or germs of any kind, in particular, bacteria, viruses, fungi, or the like.

From the EP 1 728 768 A1, for example, a system for producing an electrochemically activated saline solution by means of electrolysis is known in which a water flow charged with a saline solution or brine is supplied to an electrolysis apparatus. By electrolytic decomposition of this brine-containing water flow, an electrochemically activated aqueous saline solution can be obtained which has a comparatively high content of “free chlorine” of up to 500 mg/l, and a redox potential of +150 mlV to +1.350 mlV (as can be determined, for example, by amperometric measurements). This electrochemically activated saline solution is, owing to the comparatively high content of free chlorine, particularly convenient for use as a disinfectant, for example, for disinfecting water and/ or aqueous solutions. Furthermore, the chemically activated saline solution obtainable in accordance with the concept known from the EP 1 728 768 A1 is, according to the information given in the WO 2009/013019, also particularly convenient for use as a disinfectant in a general sense, i.e., for example, for worktops, tables, floors, for sterilisation purposes, in laundry shops or the like, or also as a carrier material in pharmaceutical substances.

Inter alia, the utilisation of such an anolyte or such an electrochemically activated saline solution having, inter alia, a free chlorine content of more than 300 mg/l as an agent for the disinfection of wounds for accelerating the healing process, or also as an agent for preventive measures, for example for hand disinfection, is also desirable. To this end, or also generally, in order to broaden the possible applications, the provision of the anolyte or electrochemically activated saline solution in a form is desirable which, on the one hand, allows for a sufficiently long retention and thus exposure time in order to achieve the desired disinfection effect while being comparatively easy to handle when applied, however, on the other hand, also enables a high microbicidal effectivity of the active agent in combination with a particularly long shelf life, i.e., in particular, high long-term stability. To this end, it is desired to dispense the electrochemically activated saline solution, i.e. the anolyte, in the form of a disinfecting foam which allows for the desired, comparatively long exposure time after application.

It is therefore the object of the invention to provide a formulation suitable for providing a disinfecting foam on the basis of an electrochemically activated aqueous saline solution enabling dispensation as a foam while preserving the microbicidal effectivity of the electrochemically activated saline solution. In addition, a foam having a particularly high microbicidal effectivity is to be provided.

With regard to the formulation, this object is, according to the invention, solved by at least one surfactant foam carrier component and at least one antimicrobially effective component on the basis of a electrochemically activated aqueous saline solution, the formulation having a content of at least 10 ppm of a free chlorine species selected from the group comprising hydrochlorous acid, hypochlorite ions, sodium hypochlorite, or dissolved chlorine gas, and exhibiting a vibration band associated with a C—F vibration in the infrared spectrum.

Advantageous embodiments of the invention are the subject matter of the dependent claims.

The invention is based on the idea that a formulation capable of providing the desired foam should contain a component on the basis of an electrochemically activated saline solution which should manifest in the formulation in the form of a measurable amount of free chlorine or a free chlorine species as a disinfecting or microbicidal component. In order to enable the dispensation in form of the desired foam including the associated prolonged possible exposure time, the formulation should, in addition, also contain a surfactant foam carrier component. In the selection of this foam carrier component, the compatibility to or combinability with the electrochemically activated saline solution should be taken into consideration to a particular degree. Particularly with regard to the factor that the formulation, where necessary, has to be kept in stock over a considerable storage period to its use after the preparation of the formulation, i.e. after combining and mixing the components, the formulation should be selected so that neither the surfactant component unduly affects the effectivity the microbicidal component, nor the microbicidal component excessively degrades or decomposes the surfactant component.

As it turned out surprisingly, such compatibility is not given for a multitude of common surfactants or foaming agent species due of the free chlorine provided as the basic component of the antimicrobially effective component. Namely, common surfactants are usually realised as hydrocarbon surfactants and, correspondingly, include hydrocarbon chains. Their C—H bonds are, in the present case, regarded as a weak spot of compatibility with the free chlorine species in the antimicrobially effective component because they might constitute a point of attack for a chlorine reaction which could lead to the decomposition of the surfactant chain and/or of the free chlorine species.

According to the invention, it is therefore contemplated to provide for the surfactant foaming agent component on the basis of fluorosurfactants. Namely, in contrast to conventional hydrocarbon surfactants as commonly also used in the detergent and personal care industry, fluorosurfactants include, in addition to a polar, hydrophilic component, a perfluorinated carbon chain which is accountable for its exceptional properties. The polar group usually renders the molecule water-soluble here while the perfluorinated chain has, in general, both hydrophobic and lipophobic properties.

The perfluorinated chain of the fluorosurfactant used according to the invention comprises a plurality of C—F bonds. The utilisation of a fluorosurfactant for providing the surfactant foam carrier component thus manifests in the infrared spectrum of the formulation and/or of the foam obtainable thereby through a vibration band associated with a C—F vibration; the use of a fluorosurfactant according to the invention is therefore, in particular, identifiable by the presence of such a vibration band associated with a C—F vibration in the infrared spectrum.

Infrared spectroscopy is a standard method of instrumental analytics for analysing and characterising atomic and molecular spectra and their structure and composition. Vibration bands associated with a C—F oscillation may be expected at frequencies or wavelengths of, for example, about 1050 cm⁻¹ or about 1250 cm⁻¹ here.

In order to achieve a particularly high antimicrobial effectivity, the formulation, in a preferred embodiment, includes a free chlorine content of more than 20 ppm, preferably of more than 300 ppm, analogously a content of an aqueous solution of more than 20 mg/l, preferably of more than 300 mg/l. It is particularly preferred here that the antimicrobially effective component is prepared by an ECA process of electrochemically activating a saline solution of the type described in the introduction. Any formulation the antimicrobially effective component of which is prepared by such an ECA process is regarded as included in the present invention and also as independently inventive here, unaffected by its demonstrability on the basis of the mentioned minimum threshold of the content of free chlorine species. It is particularly preferred here that the initial saline solution, particularly the initial saline solution prepared by the ECA process, has a free chlorine content of more than 500 mg/l, preferably of more than 600 mg/l.

Advantageously, the formulation and, in consequence, also the disinfecting foam obtainable thereby, is formulated so that it is particularly skin-compatible and environmentally friendly. To this end, the formulation preferably has a pH value of 4 to 9, preferably of 5 to 7.5.

A high antimicrobial effectivity is achievable by the formulation usefully having a redox potential of at least 200 mV, preferably of 600 to 1000 mV, preferably of 700 to 900 mV, most preferably of approximately 800 mV. In an alternative or additional advantageous embodiment, the initial saline solution has a conductivity of 12 to 16 mS/cm.

A particularly good mutual tolerance or compatibility of the antimicrobial and the surfactant component is achievable by including, in a particularly preferred embodiment, a perfluoroalkyl or a perfluoroetheralkyl as the surfactant foam carrier component. Here, it is particularly preferable to use a perfluoroetheralkyl since the perfluorinated carbon chain present therein comprises an oxygen intermediate point. It can serve as a point of attack for bacterial decomposition so that the foam formed by the formulation is biologically decomposable and therefore particularly safe for the environment.

With regard to the disinfecting foam, the mentioned object is solved by foaming a formulation according to the above explanations. Here, it is particularly preferred that the disinfecting foam exhibits a vibration band associated with a C—F vibration in the infrared spectrum.

According to one embodiment of the invention, the foam obtainable in this way is used as a disinfectant, preferably for surface or hand disinfection and/or in veterinary applications.

The advantages achieved by the invention are, in particular, that, based on an electrochemically activated initial saline solution having a sufficient content of free chlorine species for obtaining an antimicrobial effect, in combination with a surfactant foaming agent on the basis of a fluorosurfactant, a formulation can be provided in which both the antimicrobially effective and the foam forming component can be stored together without the substances decomposing or destroying each other. The foam obtainable by foaming this formulation therefore has a high antimicrobial effect, also in combination with a reliable foam formation, and even after some storage time of the formulation. Precisely by the high retention and exposure time of the antimicrobial component achievable as a result of foaming its effect as, for example, a disinfectant can be considerably increased as compared to the achievable effect of the original liquid.

One embodiment of the invention, relating to, in particular, the preparation of the formulation, will be described in detail below.

In the present embodiment, an electrochemically activated saline solution obtainable by preparation according to an ECA method and also referred to as an anolyte is used as a base substance or initial saline solution. Such an anolyte has a free chlorine content of approximately 700 mg/l, a redox potential of approximately 800 mV, a pH value of approximately 7, and an electric conductivity of approximately 14 mS/cm. Such an anolyte can be prepared electrolytically and by electrochemical activation of a saline solution in accordance with, for example, the method described in the EP 1 728 768 A1, the process parameters being appropriately selected with regard to the desired product properties. Alternatively, of course, a suitable saline solution prepared in another way could be used as the initial substance.

This electrochemically activated initial saline solution is provided with a fluorosurfactant by mixing. In the embodiment, a surfactant with the systematic name (Perfluro(2-ethanethoxy)sulphonic acid) available, for example, from Fluorochem, can be used in a suitable concentration.

1. A formulation for providing a disinfecting foam comprising at least one surfactant foam carrier component and at least one antimicrobially effective component on the basis of a electrochemically activated aqueous saline solution, wherein the formulation has a content of at least 10 ppm of a free chlorine species selected from the group comprising hydrochlorous acid, hypochlorite ions, sodium hypochlorite or dissolved chlorine gas and exhibits a vibration band associated with a C—F vibration in the infrared spectrum.

2. The formulation according to claim 1 having a free chlorine content of more than 20 mg/l.

3. The formulation according to claim 1, having a pH value of 4 to 9.

4. The formulation according to claim 1, wherein the initial saline solution has a free chlorine content of more than 500 mg/l.

5. The formulation according to claim 1 having a redox potential of at least 200 mV.

6. The formulation according to claim 1 wherein the initial saline solution has a conductivity of 12 to 16 mS/cm.

7. The formulation according to claim 1 further comprising a perfluoroalkyl or a perfluoroetheralkyl as the surfactant foam carrier component.

8. A disinfecting foam obtainable by foaming a formulation according to claim 1.

9. The disinfecting foam according to claim 8 exhibiting a vibration band associated with a C—F-vibration in the infrared spectrum.

10. The use of a foam according to claim 8 as a disinfectant, in particular for surface or hand disinfection.

11. The formulation according to claim 2, wherein the free chlorine content is more than 300 mg/l.

12. The formulation according to claim 3, wherein the pH value is 5 to 7.5.

13. The formulation according to claim 4, wherein the free chlorine content is more than 600 mg/l.

14. The formulation according to claim 5, wherein the redox potential is 600 to 1000 mV.

15. The formulation according to claim 14, wherein the redox potential is 700 to 900 mV.

16. The formulation according to claim 15, wherein the redox potential is approximately 800 mV.

17. The use of a foam according to claim 10, wherein the disinfectant is for surface or hand disinfection.
